(19) United States
(12) Patent Application Publication
(10) Pub. No.: US 2010/0063127 A1
(43) Pub. Date: Mar. 11, 2010

(54) Antioxidant Properties of Tryptophan from Human Milk

(76) Inventor: James Friel, Winnipeg (CA)

Correspondence Address: ADE & COMPANY INC., 2157 Henderson Highway, WINNIPEG, MB, R2G1P9, CA

(21) Appl. No.: 12/512,404

(22) Filed: Jul. 30, 2009

Related U.S. Application Data
(60) Provisional application No. 61/085,471, filed on Aug. 1, 2008.

(51) Int. Cl. A61K 31/405; C07D 209/42

(52) U.S. Cl. 514/415; 548/492

(57) ABSTRACT

As tryptophan is not available in sufficient amounts in infant formula or other replacement milks, we describe herein the supplementation of these milks to attain a Trp concentration similar to that of human milk. Such supplemented milks provide greater protection against free radicals.

### PRIOR APPLICATION INFORMATION

This application claims the benefit of U.S. Provisional Patent Application 61/085,471, filed Aug. 1, 2008.

### BACKGROUND OF THE INVENTION

Very low birth weight (VLBW, <1500 g birthweight) premature infants often suffer from a multitude of diseases [1,2] including chronic lung disease (CLD) or bronchopulmonary dysplasia (BPD); respiratory distress syndrome (RDS); necrotizing enterocolitis (NEC), an inflammation of the small intestine; intraventricular-periventricular hemorrhage (IVH-PVH), a brain injury often leading to developmental abnormalities; and retinopathy of prematurity (ROP), damage to blood vessels in the retina. Saugstadt [1] suggested that all problems affecting these infants are due to one unifying disease, “oxygen radical disease”, characterized by higher production and lower protection against free radicals. Also, the most important sequellae of prematurity appear to be due to inadequate protection against oxidant stress [3, 4].

In an effort to identify novel antioxidant compounds that can be used to reduce oxidative stress in these vulnerable infants, human milk (HM) samples were freeze-dried and digested with pepsin, pancreatin, and bile salts. The digested samples were filtered on a 3000 Da molecular weight cutoff membrane. The filtrate (less than 3000 Da) was separated on HPLC. The subsequent fractions were assessed for antioxidant properties through the oxygen radical absorbance capacity (ORAC) assay and both Caco-2BBE cell culture and Caco-2BBE/HT-29MTX co-culture models.

### SUMMARY OF THE INVENTION

According to an embodiment of the invention, there is provided an infant formula comprising approximately 2.2-2.4% tryptophan.

According to another embodiment of the invention, there is provided a method of treating an oxygen radical disease in a human infant in need of such treatment comprising administering to said infant an infant formula comprising approximately 2.2-2.4% tryptophan.

### BRIEF DESCRIPTION OF THE DRAWINGS

FIG. 1: ORAC values of peptide fractions (less 3000 Da) from preparative HPLC. The results are expressed as trolox equivalent per gram of sample.

FIG. 2: UV chromatogram of fraction 23 from Acquity HPLC system, column: Acquity BEH C18 1.7 uM, 2.1×100 mm, Eluent: linear gradient 0-10% acetonitrile in water for 40 min, detector: PDA at 214 nm.

FIG. 3: LC-ESI-MS of L-Trp (A) and peak at RT 9.47 in fraction #23 (B) obtained from Acquity HPLC coupled to Waters Quattro micro API mass spectrometer: LC condition as on FIG. 2; MS Condition: capillary: 3.5 kV, cone 40.0 V, Extractor 3.0 V, RF Lens 0.3 V, source temperature 100° C., desolvation temperature 200° C., cone gas flow 50 L/hr, desolvation gas flow 500 L/hr, multiplier 650 V.

FIG. 4: Online UV spectrum of standard L-Trp (A) and peak at RT 9.47 in fraction #23

FIG. 5: ORAC value of bovine whey and casein fractions, and human milk fraction 23 from preparative HPLC

FIG. 6: ORAC values of fortified and non-fortified human milk and formulas. HM: human milk, EB: Earth's Best Organic®, RSi: Similac® Alimentum® Advance®

FIG. 7: UV chromatogram at 214 nm of a plasma sample from a breastfed infant. tryptophan peak as indicated

FIG. 8: Mass spectrum of tryptophan in plasma of breastfed infant: Same condition as for the fractions of HM samples.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As used herein, ‘oxygen radical disease’ refers to a disease characterized by higher production and lower protection against free radicals. Examples of such diseases include but are by no means limited to chronic lung disease (CLD) or bronchopulmonary dysplasia (BPD); respiratory distress syndrome (RDS); necrotizing enterocolitis (NEC), an inflammation of the small intestine; intraventricular-periventricular hemorrhage (IVH-PVH); and retinopathy of prematurity (ROP).

Tryptophan (Trp) is an essential amino acid and therefore, it must be consumed in the diet as it cannot be synthesized within the body. It is not only a building block of proteins, but also functions as a biochemical precursor for several compounds, including the neurotransmitter serotonin and the neurohormone melatonin. Trp also contributes to the production of the nicotinamide moiety of co-enzymes: nicotinamide adenine dinucleotide and nicotinamide adenine dinucleotide phosphate. As discussed below, since tryptophan is not available in sufficient amounts in infant formula or other replacement milks, we describe herein the supplementation of these milks to attain a Trp concentration similar to that of human milk.

Within the human body, Trp is broken down by indoleamine and the kynurenine pathways to produce Trp metabolites. Many of these byproducts are potent free radical scavengers and thus, have powerful antioxidant capabilities. A study by Christen et al (23) described these aspects of Trp metabolism and noted that Trp metabolites may be more effective antioxidants than vitamins E and C in some respects. For example, they found that some of these metabolites were able to scavenge a greater number of free radicals (per molecule) and their antioxidant protection lasted for a longer period of time.

Few studies have examined the precise amount of Trp present in human milk. Heine (20) conducted a review of some available studies and noted that Trp is the limiting amino acid in infant nutrition, particularly in infant formulas. He indicated that the concentration of Trp in human milk protein is approximately 2.2-2.4%, while cow's milk protein contains only 1.3% Trp. The protein in infant formulas, which is derived from cow's milk, consists of approximately 1.7% Trp (20). Since α-lactalbumin has a high Trp content. Since cow's milk protein consists of only 3.7% α-lactalbumin, while human milk protein contains 29% (20), we can assume that no formula can have a higher Trp concentration than human milk.

According to the FDA, Trp is approved for the addition to infant foods. They outline several criteria that warrant the safe addition of an amino acid, including the improvement of the overall quality of the food protein. In addition, they state that the amount of Trp already present plus the amount added through supplementation cannot account for more than 1.6% of total protein. This value is lower than the level of Trp naturally present in human milk. Accordingly, one milk replacement cannot contain more Trp than another unless a company adds extra Trp or designs their milk to contain a protein ratio that is inconsistent with the FDA's recommendation (either too high or too low). As discussed below, we herein describe the addition of Trp to infant formulas up to the level present in human milk since this level is natural and thus presents no adverse effects.

According to an embodiment of the invention, there is provided an infant formula comprising approximately 2.2-2.4% tryptophan.

According to another embodiment of the invention, there is provided a method of treating an oxygen radical disease in a human infant in need of such treatment comprising administering to said infant an infant formula comprising approximately 2.2-2.4% tryptophan.

The infant in need of such treatment may be an infant suffering from or at risk of developing or showing symptoms consistent with an oxygen radical disease, for example, but by no means limited to chronic lung disease (CLD) or bronchopulmonary dysplasia (BPD); respiratory distress syndrome (RDS); necrotizing enterocolitis (NEC), an inflammation of the small intestine; intraventricular-periventricular hemorrhage (IVH-PVH); and retinopathy of prematurity (ROP). It is of note that as used herein, ‘treatment’ refers to one or more of the following: reduction in severity of symptoms, longer periods of being in remission or in a symptom-free state; lower production of free radicals, and increased protection against free radicals.

As discussed above, human milk typically contains 2.2-2.4% Trp, the benefits for which have been established as discussed above. However, in some instances, human milk may contain less than ‘normal’ levels of tryptohpan, for example, less than 2.2-2.4% Trp. In these embodiments, human milk that has less than 2.2% Trp is supplemented with Trp to an overall level of 2.2-2.4% if the level of Trp in said milk is below normal (2.2-2.4%). In addition, as will be appreciated by one of skill in the art, it is possible that the levels of tryptophan may vary during different stages of lactation and accordingly it may be necessary to determine the ‘normal’ concentration of Trp in human milk at different stages of lactation in order to facilitate safe supplementation. It is of note that in some embodiments, free Trp is used for supplementation.

Although α-amino acids, such as Trp, can occur in either L- or D-isoforms, available literature (22) suggests that the L-stereoisomer is most prevalent and biologically relevant. Furthermore, Heine (20) suggests the use of proteins with a high Trp concentration versus the use of free Trp for supplementation in infant nutrition. He indicates that the absorption of the free and protein-bound forms of Trp differs, with the latter being more readily available. He further suggests that whey protein α-lactalbumin is preferred because it contains a high concentration of Trp (5.9%).

Premature infants (<37 weeks gestation) are a very vulnerable group. These infants account for approximately 12.5% of all live births in the United States (8, 9, 10, 12). While only 1.5% and 0.7% of these infants are born at “very low” (<1500 g birthweight) and “extremely low” birth weight, respectively, they are responsible for a large proportion of all infant deaths (10). The neonatal mortality rate among late preterm infants (those born between 34-36 weeks gestation and accounting for 70-75% of all preterm births (8, 9)) is 4.6 times higher than for infants born at term (1). Those that survive are at a high risk for disability and medical complications that are extremely costly to the health care system (an estimated $26 billion per year spent on care of premature infants) (13, 14, 12). VLBW infants suffer from a multitude of diseases including: chronic lung disease (CLD) or bronchopulmonary dysplasia (BPD) and respiratory distress syndrome (RDS); patent ductus arteriosus (PDA); necrotizing enterocolitis (NEC), an inflammation of the small intestine; intraventricular-periventricular hemorrhage (IVH-PVH) a brain injury often leading to developmental abnormalities; and retinopathy of prematurity (ROP), which involves damage to blood vessels in the retina. Of the premature infants who survive long enough, 20% develop BPD (11), 65% develop PDA, 5-10% suffer from NEC (for which the mortality rate is 15-30%), and those born <28 weeks gestation are 18 times more likely to develop ROP than those born >28 weeks gestation (10).

They are also at risk of abnormal development due to high inspired oxygen concentrations, among other factors. Due to immature lung development, immediately after birth, premature infants do not get enough oxygen (hypoxia) and, therefore, require supplemental oxygen in concentrations as high as 95%. Since the levels of inspired oxygen required to maintain arterial oxygen tension in post-natal life are substantially higher than those present in the womb, premature infants are exposed to more reactive oxygen species (ROS) than if they had remained in utero. Saugstadt (1) suggests that all the factors, conditions and problems affecting premature infants are the outcome of one unifying disease, “oxygen radical disease”. He suggests that there is higher production and lower protection against free radicals. Clearly there is a need to reduce oxidative stress and/or boost antioxidant defenses in these vulnerable infants. Data from our group (15) and others (3, 16, 17, 18) suggests that HM has antioxidant properties that will assist the premature in coping with increased oxidative stress.

In an effort to identify novel antioxidant compounds that can be used to reduce oxidative stress in these vulnerable infants, human milk (HM) samples were freeze-dried and digested with pepsin, pancreatin, and bile salts. The digested samples were filtered on a 3000 Da molecular weight cutoff membrane. The filtrate (less than 3000 Da) was separated on HPLC. The subsequent fractions were assessed for antioxidant properties through the oxygen radical absorbance capacity (ORAC) assay and both Caco-2BBE cell culture and Caco-2BBE/HT-29MTX co-culture models, as discussed herein. As will be well known to one of skill in the art, Oxygen Radical Absorbance Capacity (ORAC) refers to the free radical quenching capacity or antioxidant capacity of a compound. Regarding breast-milk, higher antioxidant capacity values provide greater protection against oxidative stress in an infant (19).

### Milk Sample

The sample (40 L) used was a mature milk obtained from a volunteer mother who did not breastfeed her infant. The sample was stored by the mother in −20° C. freezer and transported to our laboratory on dry ice. Once in our laboratory, the milk was freeze dried and stored at −80° C. until ready for analysis. We repeated the analysis on 2 other milk samples form volunteer mothers to confirm that this was not just a finding from one mother that could be due to dietary influences. We found the same analytical profile in all 3 samples form 3 different mothers.

### Digestion Protocol

The solid content of human milk is about 13% [5]. Based on this composition, 130 g of freeze-dried HM was diluted with 870 mL of deionized water. The digestion model mimicking premature infant digestion was modified from that utilized by Etcheverry et al [6]

Gastric digestion: To mimic the gastric digestion of the premature infant, the sample was further diluted with physiological concentrations of salts [6] (530 mL of 140 mM NaCI plus 5 mM KCI). The pH was adjusted to 5.5 with 1 M HCI, then 76 mL of pepsin (Sigma P7000, 4 g in 100 mL of 0.1 M HCI) was added, and the final pH was adjusted to 4.0. The sample was incubated for 30 min at 37° C., 100 rpm on a MaxQ 4000 incubator (Barnstead Lab-Line) then adjusted to pH 6.0 and incubated for another 30 min.

Intestinal digestion: After gastric digestion, 380 mL of pancreatin (Sigma P1730, 0.8 g) and bile salts (Sigma B3883, 4.8 g) were diluted in 400 mL of 0.1 M NaHCO₃. The pH was adjusted to 7.0 with 1 M NaHCO₃ and incubated for 2 hrs at 37° C. The sample solution was heated in a water bath at 90° C. for 15 min to inactivate the enzymes (pancreatin and bile salts).

Removal of lipid: The digested HM sample was stored at +4° C. overnight in a separatory funnel to separate the lower peptide-containing phase from the upper lipid-containing phase.

### Membrane Filtration

An Amicon stirred cell unit 8400 and a 3000 Da molecular cutoff membrane (Millipore, Billerica, Mass.) was used to separate the peptides into low and high molecular weight (>3000 Da). The low molecular weight filtrate (<3000) was freeze dried and then stored at −80° C. for preparative HPLC separation.

HPLC Separation

Column: Symmetry 300 C18 (5 μm 19×250 mm) from Waters Corp.
Solvent: Linear gradient 0-70% B in 60 min at a flow rate of 6.0 mL/min; A: 0.05% TFA in water, B: 0.5% TFA in acetonitrile. Detector waters PDA 996 set at 214 nm; HPLC system: Waters 600E Multisolvent Delivery System; Fraction collection: the Waters fraction collector III was used to collect the fraction every min (6 ml) and a total of 55 fractions were collected and combined from different runs. These fractions were freeze dried and used for ORAC and intracellular ROS assays.

### Oxygen Radical Absorbance Capacity (ORAC)

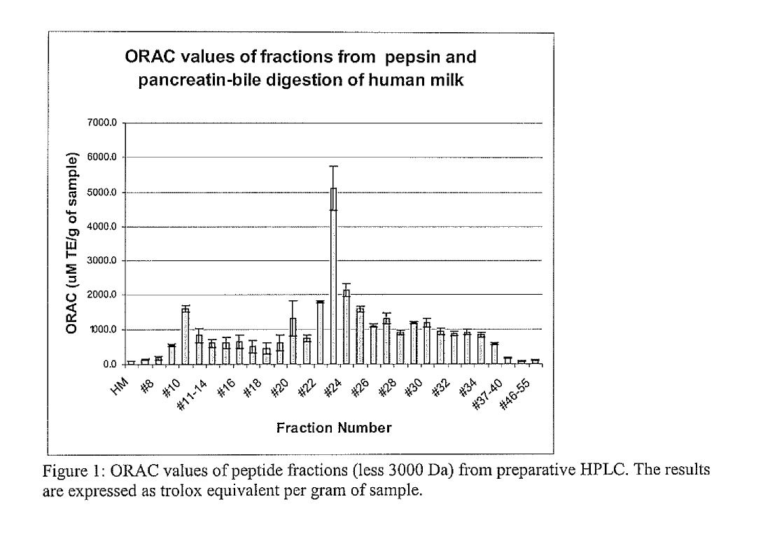

This assay measures the scavenging capacity of antioxidants in nutrients or in vivo against the peroxyl radical [7], which is one of the most common reactive oxygen species (ROS) found in the body. Out of all fractions collected (FIG. 1), fraction #23 was found to have the highest ORAC value (5116±150 uM Torlox eq/g sample), which was 65 times higher than the whole HM sample. To identify compounds in #23, the sample was injected into a LC-MS/MS system.

### Compound Identification

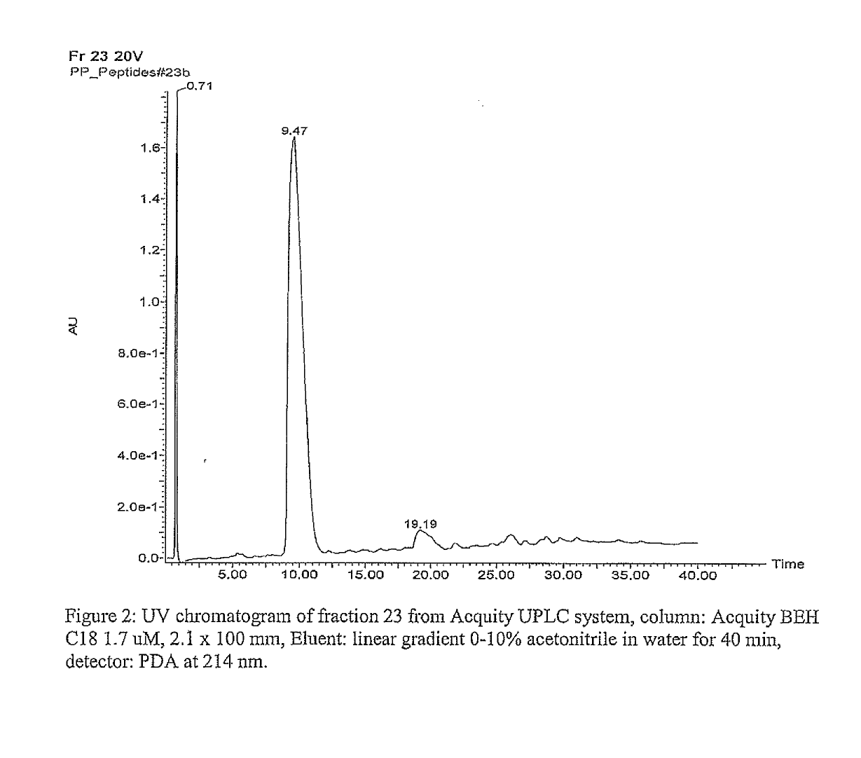

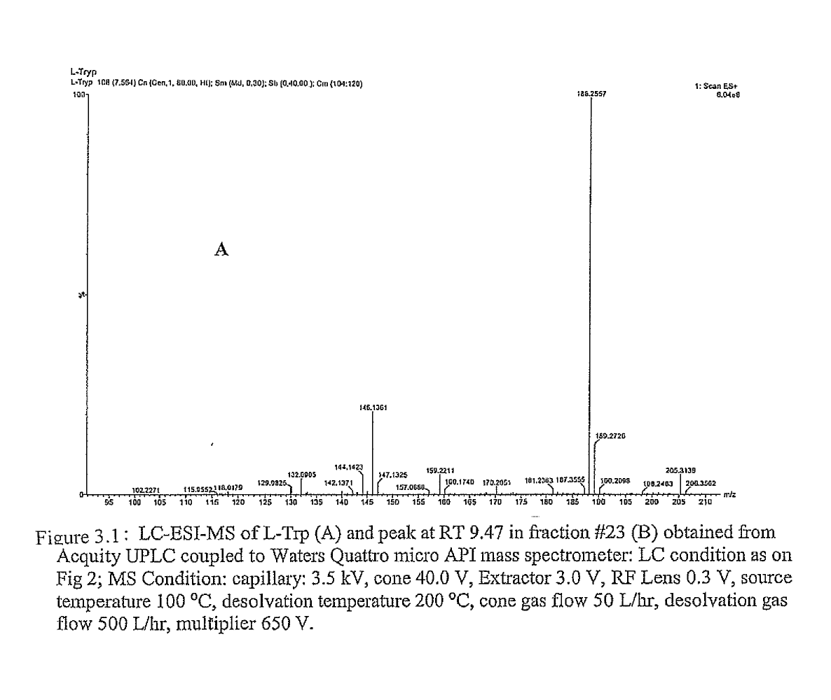

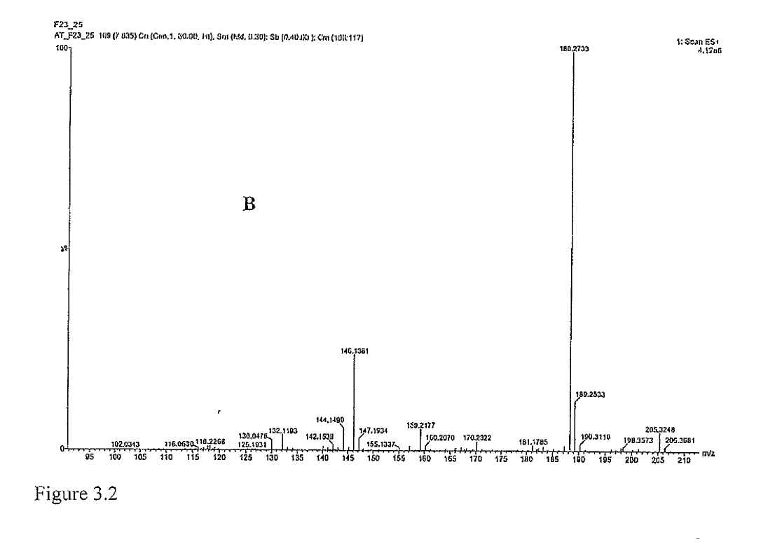

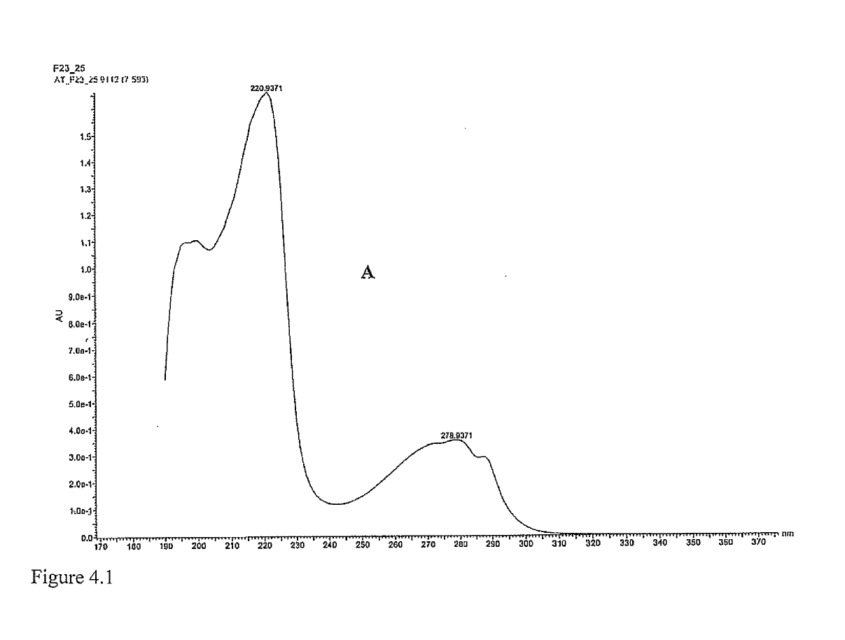

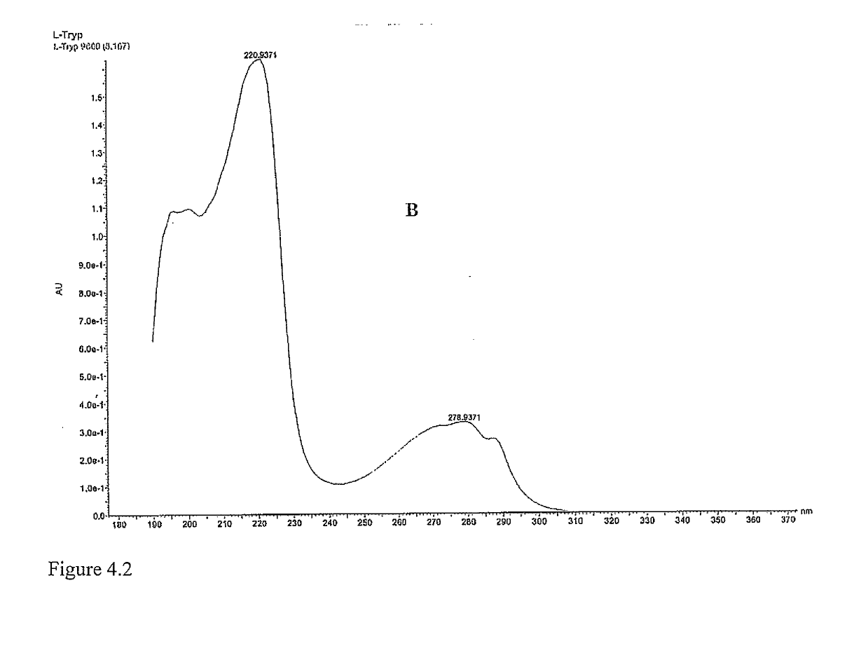

The active fraction 23 (1 mg/ml) was injected into the Waters Acquity™ HPLC system coupled to a Waters Micromass Quattro Micro API, as well as to a Maldi Q-TOF mass spectrometer. The UV chromatogram of fraction 23 (FIG. 2) showed one large peak at retention RT 9.47 while the mass spectrum (FIG. 3) showed that it contained about fifteen minor components.

The major peak at RT 9.47 appeared to be the major antioxidant compound in fraction #23. LC-MS/MS analysis showed that this compound is the essential amino acid tryptophan. This was confirmed by LC-MS/MS data from #23 and by analyzing standard tryptophan purchased from Sigma (FIG. 3).

Cow's Milk Doesn't have the Same Amount of Tryptophan after Digestion

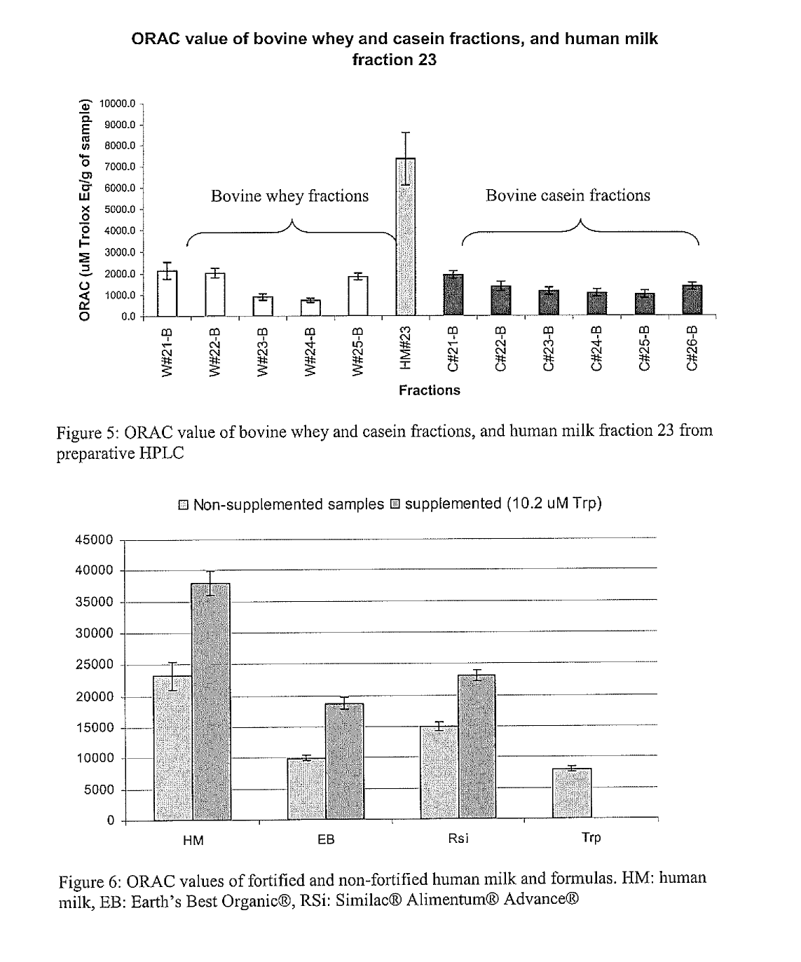

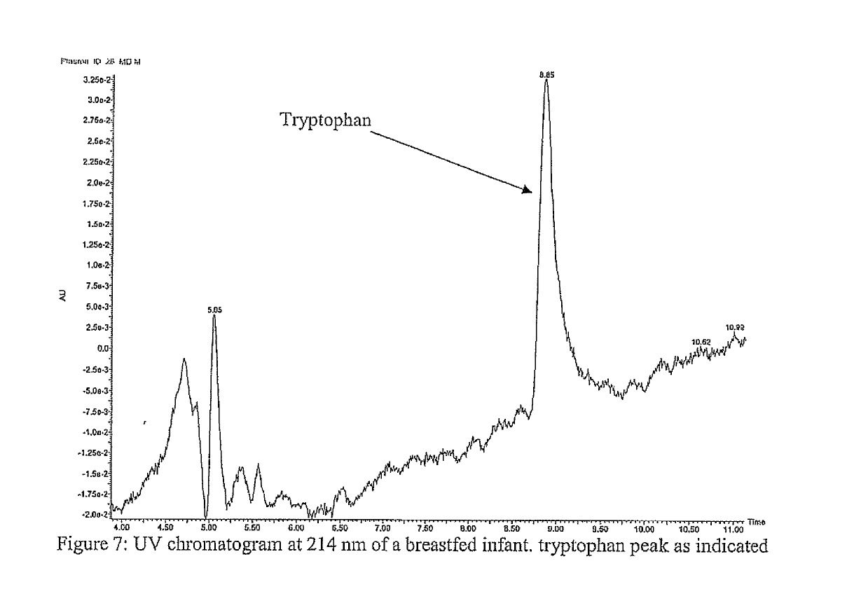

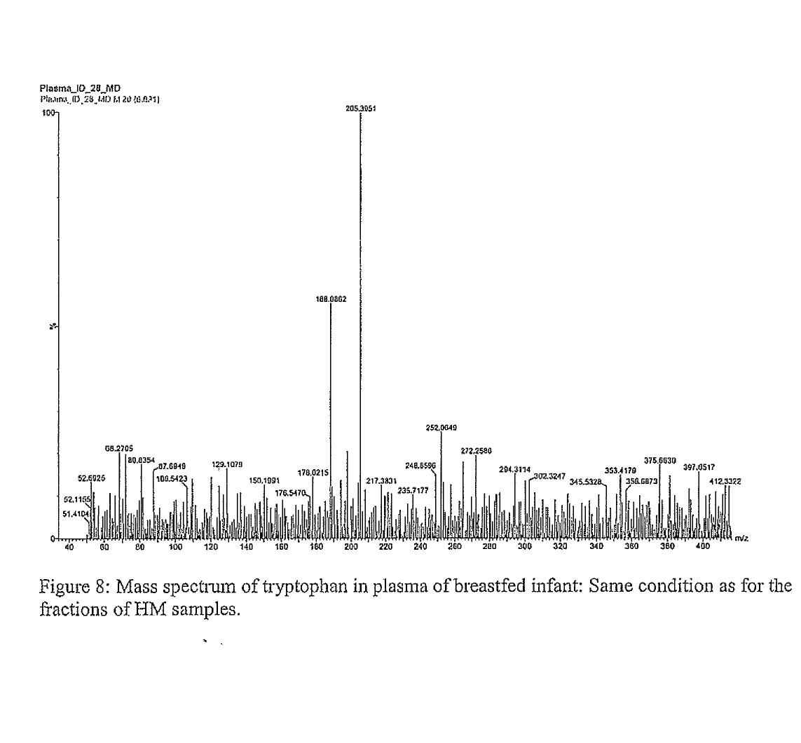

Whey and casein protein purchased from Sigma were digested in a similar manner as the human milk. The fractions were concomitantly evaluated for their antioxidant activity using the ORAC assay. Fraction #23 from human milk showed a higher antioxidant activity (FIG. 5).

Supplementation of Human Milk and Formula with Tryptophan

Antioxidant activity of human milk and two infant formulas were evaluated before and after supplementation with tryptophan. The final concentration of supplemented tryptophan in both human milk and formula was 10.2 μmol/L. The ORAC assay showed that HM contained the most active form of tryptophan and that the effect of supplementation was additive (Table 6). Specifically, HM had the highest ORAC and when we added Trp it increased. ORAC values of both Trp supplemented HM and Formulas increased.

### Tryptophan is Present in Baby Plasma

A blood sample was collected in EDTA tube from one month-old breastfed infant. The sample was centrifuged at 3000 g, 4° C. to separate plasma and red blood cells. One-hundred microlitres of 10% TCA (trichloroacetic acid) was added to 100 μL of plasma and the mixture was centrifuged at 10,000 rpm for 10 min at room temperature. The sample was filtered on a 0.45 μM Millipore membrane and injected into LC-MS/MS for analysis. Both the LC chromatogram and mass spectrum showed the presence of tryptophan in the plasma of the breastfed infant.

### Rationale

Fraction 23 has the highest ORAC value and also significantly reduces oxidative intracellular ROS.

Tryptophan have been identified by LC-MS/MS as the main compound in #23. While not wishing to be bound to a particular hypothesis or theory, the inventors believe that Trp helps protect infants against oxidative stress by: 1) scavenging the free radical directly, or 2) crossing the cells and up-regulating the antioxidant response elements.

While the preferred embodiments of the invention have been described above, it will be recognized and understood that various modifications may be made therein, and the appended claims are intended to cover all such modifications which may fall within the spirit and scope of the invention.

TABLE 1
Effect of Tryptophan on TNFα Expression

| | TNFα(pg/ml) |
|---|---|
| Medium + LPS 1 μg/ml | 6156 |
| Tryptophan 50 μg/ml | 4869 |

This was carried out using a cell based immunoassay using mouse monocyte/macrophage cells (RAW 264.7; ATCC American Tissue Culture Collection). The experiment was done in triplicates. The data were analyzed by t-test. A significant difference was found between the control value and the treated value ay p<0.05. Based on this tryptophan is an ant-inflammatory amino acid.

### REFERENCES

1. Saugstadt OD. Oxygen toxicity in the neonatal period. Acta Pediatr Scand 1990; 79: 881-92.
2. Sirven V, Beck G, Chrostowski D, Cardenas P, Boxer H, Frieri M. Monitoring circulating nitric oxide levels in infants with bronchopulmonary dysplasia for disease activity. Curr Ped Rev 2005; 1: 91-5
3. van Zoeren-Grobben D, Lindeman J H, Houdkamp E, Brand R, Schrijver J, Berger H M. Postnatal changes in plasma chain-breaking antioxidants in healthy preterm infants fed formula and/or human milk. Am J Clin Nutr 1994; 60: 900-6.
4. Saugstadt O D. Oxygen radical disease in neonatology. Semin Neonatol 1998; 3: 239-44
5. Shehadeh N, Aslih N, Shihab S, Werman M J, Sheinman R, Shamir R. Human Milk Beyond One Year Post-partum: Lower Content of Protein, Calcium, and Saturated Very Long-chain Fatty Acids, The J Pediatrics 2006; 148:122-124.
6. Etcheverry P, Wallingford J C, Miller D D, Glahn R P. Calcium, zinc, and iron bioavailabilities from a commercial human milk fortifier: a comparison study. J Dairy Sci 2004; 87:3629-3637
7. Ou B, Hampsch-Woodill, Prior, R. Development and validation of an improved oxygen radical absorbance capacity assay using fluorescein as the fluorescent probe. J Agric Food Chem 2001; 49:4619-4626
8. Engle W A, Kominiarek M A. Late preterm infants, early term infants, and timing of elective deliveries. Clin Perinatol 2008; 35: 325-341.
9. McIntire D D, Leveno K J. Neonatal mortality and morbidity rates in late preterm births compared with births at term. Obstetrics & Gynecology 2008; 111(1): 35-4
10. Eichenwald E C, Stark A R. Management and outcomes of very low birth weight. N Engl J Med 2008; 358 (16): 1700-11.
11. Baraldi E, Filippone M. Chronic lung disease after premature birth. N Engl J Med 2007; 357: 1946-55.
12. Institute of Medicine of the National Academies. Preterm birth: Causes, consequences, and prevention. Retrieved on 7 Jul. 2008 from: http://www.iom.edu/CMS/3740/25471/35813.aspx
13. Barsky V, Siegel L. Predicting future cognitive, academic, and behavioural outcomes for very low birth weight infants. In S. Friedman & M. Sigman (Eds.), The psychological development of low birth weight infants 1991: pp. 275-298. Norwood, N.J.: Ablex.
14. Vohr B, Marshall M. Neurological and functional outcomes of very low birthweight infants. Seminars in Primatology, 1997:21; 202-220.
15. Friel J K, Martin S M, Langdon M, Herzberg G and Buettner G R. HM provides better antioxidant protection than does infant formula. Pediatric Research 2002: 51:612-618.
16. Shoji H, Shimizu T, Shinohara K, Oguchi S. Suppressive effects of breast milk on oxidative DNA damage in very low birthweight infants. Arch Dis Child Fetal Neonatol 2004:89F136-F138.
17. Goldman A S, Thorpe L W, Goldblum R M, Hanson K A. Anti-inflammatory properties of human milk. Acta Pediatr Scand 1986:75:689-695.
18. Buescher E S McIllherhan S M. Antioxidant properties of human colostrum. Pediatr Res 1988; 24:14-19.
19. Shoji H, Koletz B. Oxidative stress and antioxidant protection in the perinatal period. Curr Opin Clin Nutr Metab Care 2007; 10:324-8.
20. Heine WE. The significance of tryptophan in infant nutrition. Huether et al, Eds. Tryptophan, Serotonin, and Melatonin: Basic Aspects and Applications. New York: Kluwer Academic/Plenum Publishers, 1999.
21. Ishi Y, 2007
22. Nelson, Cox. 2005
23. Christen S, Peterhans E, Stocker R. Antioxidant activities of some tryptophan metabolites: Possible implication for inflammatory diseases. Proc Nat Acad Sci 1990; 87; 2506-2510.

1. An infant formula comprising approximately 2.2-2.4% tryptophan.

2. A method of treating an oxygen radical disease in a human infant in need of such treatment comprising administering to said infant an infant formula comprising approximately 2.2-2.4% tryptophan.
